(19) United States
(12) Patent Application Publication
(10) Pub. No.: US 2026/0249082 A1
(43) Pub. Date: Aug. 27, 2026

(54) IMPLANTABLE MEDICAL DEVICE WITH T-WAVE PROTECTION

(71) Applicant: BIOTRONIK SE & Co. KG, Berlin (DE)

(72) Inventor: Andreas KUTSAM, Berlin (DE)

(73) Assignee: BIOTRONIK SE & Co. KG, Berlin (DE)

(21) Appl. No.: 19/489,727

(22) PCT Filed: May 31, 2024
(86) PCT No.: PCT/EP2024/065026

§ 371 Date: Dec. 3, 2025

(30) Foreign Application Priority Data
Jun. 9, 2023 (EP) 23178451.3

(51) Int. Cl. A61N 1/362; A61N 1/365; A61N 1/368

(52) U.S. Cl. CPC A61N 1/3627; A61N 1/36514; A61N 1/36843

(57) ABSTRACT

An implantable medical device, including a left ventricular, LV, electrode assembly for connection to the myocardium of the left ventricle of a patient's heart and configured for application of left ventricular pacing, LVp, and for left ventricular sensing, LVs, and a control unit, configured to control the LV electrode assembly with respect to an application of LVp at a scheduled LV stimulation time, the control including inhibiting LVp during a left ventricle upper rate interval, LVURI, for T-wave protection, wherein a start of the LVURI is triggered by an LVs or LVp event. The control unit is further configured, if the scheduled LV stimulation time falls into the LVURI, to apply LVp when the LVURI lapses.

### CROSS-REFERENCE TO RELATED APPLICATIONS

This application is the United States National Phase under 35 U.S.C. § 371 of PCT International Patent Application No. PCT/EP 2024/065026, filed on May 31, 2024, which claims the benefit of European Patent Application No. 23178451.3, filed on Jun. 9, 2023, the disclosures of which are hereby incorporated by reference herein in their entireties.

### TECHNICAL FIELD

The invention refers to an implantable medical device comprising a left ventricular, LV, electrode assembly and a control unit. The invention particularly refers to a heart stimulator intended to administer cardiac resynchronization therapy (CRT). Such a heart stimulator can embody an implantable cardiac pacemaker or similar functionality as enabled via implantable cardiac cardioverter/defibrillator devices.

### BACKGROUND

By its LV electrode assembly, the medical device provides electrical stimulation pulses to the myocardium, facilitating a contraction of the left heart chamber. The stimulation pulses are applied in accordance with a timing regimen to achieve left ventricular pacing, LVp. The LV electrode assembly is further configured for LV sensing, LVs, to sense left ventricular events events.

LVp leads to a depolarization of the myocardium causing a contraction of the LV. When the myocardium is fully depolarized it is in a refractory period in which it is unsusceptible to further excitation. Thereafter, the myocardium repolarizes and relaxes so that the ventricle can expand. In a typical intracardiac electrogram, depolarization of the myocardium corresponds to the “R-wave”. The repolarization of the myocardium coincides with the “T-wave”.

LVp application during the T-wave may induce ventricular tachycardia, VT, or ventricular fibrillation, VF. Hence, the medical device is configured to inhibit LVp during a left ventricle upper rate interval, LVURI, which is started by LVp and LVs events. LVURI is a programmable time interval which should be sufficiently long to cover the length of the T-wave and realize an efficient LV T-wave protection.

It is known that inhibiting LVp by LV T-wave protection may lead to a lock-in phenomenon at intrinsic intervals that are sufficiently close to the length of LVURI, e.g., during a period of increased activity of the patient resulting in elevated heart frequencies approximately above 100 bpm.

Such an LVURI lock-in may proceed as follows: At first, an LVs event triggers the start of an LVURI. The LVs event may be caused by an extra systole, an oversense of the P-wave with the LV electrode assembly or a measurement sequence carried out by the medical device. Then, the LVURI lock-in may start when a scheduled LVp is inhibited because it falls into the LVURI. After lapse of the LVURI and before the next scheduled LVp, an LVs event, for example, caused by an intraventricular conduction of a stimulation pulse of a right ventricular, RV, electrode assembly, RVp, to the LV, or the intrinsic conduction from the atrium to the LV, triggers the start of another LVURI which, again, causes inhibition of the next scheduled LVp so that the LVURI lock-in continues and may be perpetuated by intraventricular conduction of RVp stimulation pulses.

An LVURI lock-in perpetuated by LVs events caused by RVp, or intrinsic atrium to LV conduction, may only end if the heart rate is decreased to a rate low enough that the planned LVp does not fall within the LVURI anymore.

An LVURI lock-in has a clinically relevant impact on the patient that can culminate in a decompensated cardiac failure (Alasti, M. et al., Journal of arrhythmia, 33(6), 652-654, 2017). This makes it desirable to avoid or at least break the perpetuation of an LVURI lock-in. A straightforward solution to avoid the LVURI lock-in is to not inhibit LVp, i.e., not to use an LVURI at all. This comes at the expense of losing LV T-wave protection and risking induced arrhythmias. Another known solution to reduce the probability of occurrence of LVURI lock-in is reducing the length of LVURI. Such an approach is limited because LVURI should not be shorter than 375 ms to guarantee LV T-wave protection.

The present disclosure is directed toward overcoming one or more of the above-mentioned problems, though not necessarily limited to embodiments that do.

### SUMMARY

It is, therefore, an object of the present invention to provide an improved medical device configured to inhibit LVp during an LVURI for T-wave protection.

According to a first aspect of the invention, this object is achieved by providing a medical device according to claim 1.

Accordingly, an implantable medical device is provided that comprises an LV electrode assembly for connection to the myocardium of the left ventricle of a patient's heart and configured for application of LVp and configured for LVs. The implantable medical device further comprises a control unit, configured to control the LV electrode assembly with respect to an application of LVp at a scheduled LV stimulation time, the control including inhibiting LVp during an LVURI for T-wave protection, wherein a start of the LVURI is triggered by an LVs or LVp event. The control unit is further configured, if the scheduled LV stimulation time falls into the LVURI, to apply LVp when the LVURI lapses.

The LV electrode assembly being configured for application of LVp may comprise a configuration for applying stimulation pulses in accordance with a timing regimen. The electrode assembly being configured for LVs may comprise the electrode assembly being configured to detect electrical LV events such as an intrinsically induced contraction of the myocardium.

The scheduled LV stimulation time may be a predetermined time at which an LV stimulation is scheduled to be applied. A series of scheduled LV stimulation times may realize pacing at a desired LV rate interval. The start of the LVURI may not only be triggered by LVs but also be triggered by LVp so that after a stimulation signal has been applied to the myocardium of the LV, further LVp during the LVURI is inhibited. The LV rate interval is preferably longer than the LVURI. In such a case, a scheduled LV stimulation time may only fall into the LVURI if the start of the LVURI is triggered by LVs (not LVp).

According to the configuration of the control unit, the control unit may check, whether an LV stimulation time is scheduled within the LVURI. Since LVp at such a scheduled LV stimulation time is inhibited for reasons of T-wave protection, the accordingly scheduled LVp is normally skipped. By applying LVp when the LVURI lapses, the control unit may be understood to be configured to compensate for the skipped LVp during LVURI at the end of LVURI. With such a configuration, it is possible to achieve a biventricular pacing or LV only pacing, instead of pacing only in the RV, as it would occur if LVp would be skipped.

Lapsing of the LVURI may be understood as a full length LVURI coming to an end. In a further embodiment, start of a new LVURI is triggered by an LVs event that falls into an on-going LVURI. The current LVURI may be understood to be restarted. In such an embodiment, lapsing of the LVURI means lapsing of the new LVURI. Analogously, a series of overlapping LVURIs may lapse with its last LVURI coming to an end.

In one embodiment, the medical device comprises a right ventricular, RV, electrode assembly for connection to the myocardium of the right ventricle of the patient's heart and configured for application of right ventricular pacing, RVp. The stimulation pulse applied by the RV electrode assembly to achieve RVp may travel from RV to LV where it may cause an LVs event. The LVs event may trigger a start of the LVURI. If the LV rate intervals are sufficiently short (which is the case at an increased intrinsic rate, for example) and an LVs event caused by RVp is the first LVs event after the LVURI, repeating LVURIs would continuously inhibit LVp. However, since the control unit is configured to apply LVp when the LVURI lapses, the LVp may start an earlier LVURI at a time before an LVs event caused by RVp. By coupling LVp to the lapse of LVURI in such a way, biventricular pacing may be achieved.

In one embodiment, the control unit is configured to control the RV electrode assembly with respect to an application of RVp at a scheduled RV stimulation time which is delayed by a ventricular-ventricular, VV, time interval with respect to the scheduled LV stimulation time so that LVp comes first, wherein the VV time interval is, in particular, 5 to 100 ms, 5 to 90 ms, 10 to 80 ms, 10 to 70 ms, 10 to 60 ms, 10 to 50 ms, or 10 to 40 ms.

RVp may generally be applied at a scheduled RV stimulation time by the RV electrode assembly. Additionally, the VV time interval may pass between the scheduled RV stimulation time and the scheduled LV stimulation time. The delay introduced by the VV time interval may realize an LV first stimulation. Hereby, the VV time interval may be used to compensate an intrinsic VV delay as follows: After LVp, the induced electrical stimulation spreads out within the myocardium. When the stimulation is expected to have reached the RV, i.e., after the intrinsic VV delay is expected to have been passed, the VV time interval may be programmed to simultaneously lapse and R Vp may be applied. The VV time interval may be programmable between 5 to 100 ms. In particular, it may be adjusted to 10 to 40 ms. The intrinsic VV delay may be prolonged if the patient suffers from a left bundle-branch block (LBBB). Alternatively, both ventricles may be paced simultaneously (corresponds to a VV time interval of 0 ms).

The LBBB may also cause the LVs to be delayed relative to an RV sensing, RVs. Typically, such an LBBB delay may be 40 to 200 ms or, more specifically, 70 to 130 ms. A stimulation spreading from the sinus node or from atrial pacing towards the ventricles may cause an LVs event after an RVp event (delayed by the LBBB delay). Such a delayed LVs may trigger the start of an LVURI that continues an LVURI lock-in.

In one embodiment, the LVURI is less than or equal to an RV upper tracking interval, RVUTI, plus the VV time interval. This may ensure that a desired timing, wherein the LVp is applied by the VV time before the RVp can be restored. If the LVURI is longer in time than the RVUTI plus VV time, a scheduled LV stimulation time may always overlap with an LVURI.

In one embodiment, the medical device comprises an atrial electrode assembly for connection to the myocardium of the right atrium of the patient's heart and configured for atrial sensing, As, and/or atrial pacing, Ap, wherein the scheduled LV stimulation time is calculated by the control unit based on a timing of the respective atrial event. Depending on the desired operating mode of the medical device, the control unit may provide a different timing as well or at least calculate the timing independent of atrial events.

Together, the atrial electrode, the RV electrode and the LV electrode may form a three chamber system. Either of As or Ap may trigger a start of a programmable arterial ventricular delay, AVD, interval. The AVD interval may mimic the AVD which is caused by the spreading time of the natural sinus node stimulation from the atrium to the ventricles. A natural contraction pattern may be achieved by applying the AVD interval. For example, the RVp may be applied, when the AVD interval lapses. The LVp may be applied at the same time or within an interval of, for example 100 ms before and 100 ms after the scheduled RVp time. Alternatively, the LVp may be applied within an interval of for example 150 ms to 100 ms before and 100 ms to 150 ms after the scheduled RVp time. An example calculation of the scheduled LV stimulation time may start from an As event, wherein the AVD interval is added to the time of the As event in order to calculate a scheduled RVp stimulation time. From the scheduled RVp time, the VV time may be subtracted to calculate the scheduled LV stimulation time.

In one embodiment, the control unit is further configured, if the scheduled LV stimulation time falls into the LVURI, to apply LVp when the LVURI lapses only if a lapse time of the LVURI is within a predetermined time interval that includes the scheduled RV stimulation time. The predetermined interval may start an interval of the VV time before and end 150 ms, in particular 100 ms, after the scheduled RV stimulation time. Such coupling of the LVp to the RVp may ensure that the (additional) LVp applied when the LVURI lapses cannot happen at any arbitrary point in time of the heart's activity.

For example, if the start of an LVURI is triggered by an oversense of a P-wave at the LV electrode assembly at a (low) heart rate of 60 bpm, a first LVURI may be triggered to start so that a scheduled LVp is skipped. The first LVURI may lapse, for example, after 400 ms. Before the first LVURI can lapse, however, an LVs event caused by RVp may trigger a second LVURI. For example, the RVp may be scheduled at an AVD time of 140 ms after the P-wave, wherein, firstly, the scheduled RV stimulation time may be calculated by the control unit based on a time of an atrial sense, As, and, secondly, an intraventricular delay, IVD, time between the RVp and LVs may be 100 ms so that an LVs occurs 240 ms after the P-wave which is before the first LVURI has lapsed after 400 ms. Now, the second LVURI may lapse after 400 ms which is 640 ms after the P-wave. If an LVp is applied 640 ms after the P-wave, it may be between a T-wave and the next P-wave so that its application may disturb the heart's rhythm. By coupling the LVp application to the predetermined time interval that has the RV stimulation time within, such a situation may be avoided. The time interval may extend, for example, up to 250 ms.

In one embodiment, the LVURI is a constant that is programmable between 375 to 667 ms (corresponds to 90 to 160 bpm). Preferably, LVURI may be programmed to a value between 375 to 462 ms (corresponds to 130 to 160 bpm), for example, 462 ms (corresponds to 130 bpm) or 400 ms (corresponds to 150 bpm).

According to a second aspect, the object is also achieved by a method for operating an implantable medical device, comprising the steps of sensing, by an LV electrode assembly of the medical device for connection to the myocardium of the left ventricle of a patient's heart, an event, LVs; triggering, by a control unit of the medical device, a start of an LVURI for T-wave protection, during which an application of LVp through the LV electrode assembly is inhibited; and if a scheduled LV stimulation time falls into the LVURI, applying, by the control unit, LVp when the LVURI lapses.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea of the invention shall subsequently be described in more detail with reference to the embodiments as shown in the drawings. Herein:

FIG. 1 shows a schematic diagram of timing cycles of As, RVp and LVs with a prolonged LVURI lock-in; and

FIG. 2 shows a schematic diagram of timing cycles of As, RVp and LVs with a resolved LVURI lock-in.

### DETAILED DESCRIPTION

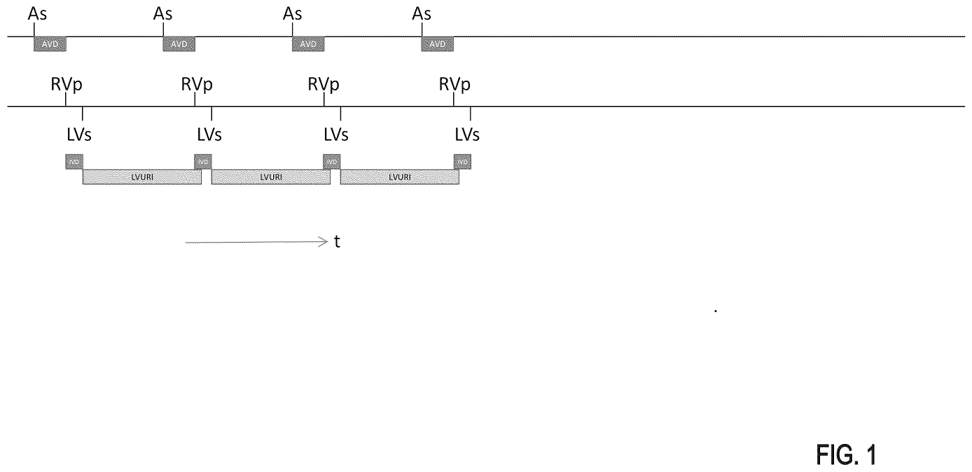

FIG. 1 shows a series of atrial sensing, As, events spaced apart by a constant time interval that represents the natural sinus rhythm (top panel of FIG. 1). The As events are sensed by an atrial electrode assembly of a medical device connected to the myocardium of the right atrium of a patient's heart and configured for atrial sensing. The atrial electrode assembly may also be configured for atrial pacing.

The natural sinus rhythm induces an electrical stimulation that travels from the sinus node to the ventricles, causing a contraction of the ventricles within an atrio-ventricular delay, AVD, in a healthy patient. In order to mimic such a natural behavior, the medical device comprises a right ventricular electrode assembly connected to the myocardium of the right ventricle of the patient's heart and configured for application of right ventricular pacing, RVp. Such RVp is applied (middle panel of FIG. 1) by the medical device after a (predetermined) AVD time interval has lapsed. Spacing between RVp events is limited by an upper tracking interval, UTI, of the medical device which is typically within 353 to 667 ms (corresponds to an upper tracking rate, UTR, of 90 to 170 bpm). That is, the spacing between adjacent RVp events is normally above 353 ms and the medical device may be configured to not support faster pacing. Preferably, the UTI is 400 to 500 ms (corresponds to 120 to 150 bpm), for example, the UTI can be 462 ms (corresponds to 130 bpm).

The first RVp event comes after an AVD time after the first As event and induces an electrical stimulation that spreads from the RV to the LV within an intraventricular delay, IVD. The IVD may be 10 to 200 ms or more specifically 70 to 150 ms. The medical device further comprises a left-ventricular electrode assembly connected to the myocardium of the left ventricle of the patient's heart and configured for application of left ventricular pacing and for left ventricular sensing. The electrical stimulation spreading from the RV to the LV causes a first LVs event following the first RVp event (bottom panel of FIG. 1).

A control unit of the medical device is configured to control the LV electrode assembly with respect to an application of LVp at a scheduled LV stimulation time, the control including inhibiting LVp during a left ventricle upper rate interval, LVURI, for T-wave protection. The first LVs event triggers the start of a first LVURI. If an LVp had been applied before the RVp, start of an LVURI would already have been triggered for the IVD time plus an optional ventricular-ventricular, VV, time, the latter of which indicates by how much time the LVp is scheduled to come before the RVp.

Another LVp is scheduled to be applied after the second As event and before the second RVp event. This timeslot falls into the LVURI so that LVp is inhibited. The second RVp event causes a second LVs which triggers the start of a second LVURI. Since the timing pattern does not change for the next two RVp events, no LVp can be applied at its scheduled LVp time—LVp is locked in by recurring LVURIs, a phenomenon known as LVURI lock-in. The LVURI lock-in may be resolved when the distance between adjacent RVp events is increased, i.e. in this case with pacing based on As events by a lower intrinsic rate.

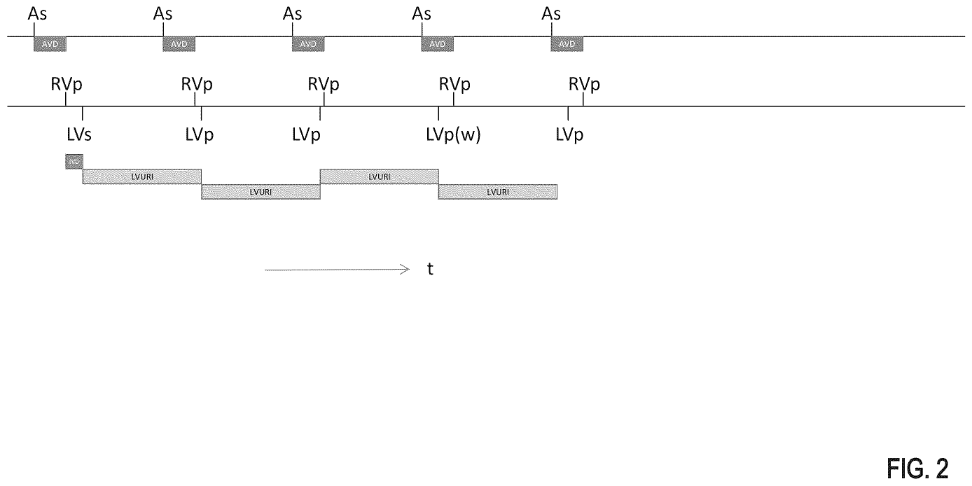

In FIG. 2, under identical initial conditions as in FIG. 1 (first RVp and following LVs causing LVURI lock-in), LVp is applied when the first LVURI lapses. The (first) LVp stimulation time is before the second RVp stimulation time plus the IVD so that the first LVp triggers the start of the second LVURI before an LVs event happens. Since the LVURI is less than the distance (in time) between adjacent RVp events in order to deliver a subsequent LVp to a point in time which is closer to the intended LVp time according to the CRT therapy, the second LVURI lapses even earlier and before the third RVp stimulation time plus IVD. The third LVURI lapses earlier again and much more before the fourth RVp stimulation time plus IVD. The fourth LVURI lapses even before the scheduled LVp stimulation time so that an LVp is applied as scheduled and the desired timing where an LV stimulation time is a VV time before an RV stimulation time is restored.

By applying LVp when the LVURI lapses if the scheduled LV stimulation time falls into the LVURI, a desired timing of stimulation events may be restored within a small number of stimulation events. Specifically, the number of stimulation events required to restore the desired timing of stimulation events may depend on the individual patient's IVD and the (predetermined) VV time which describes a delay of a scheduled RV stimulation time with respect to a scheduled LV stimulation time and the current pacing rate (RVp rate). Thus, a biventricular pacing with the desired timing regimen may be quickly restored after an LVURI lock-in.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An implantable medical device, comprising
a left ventricular, LV, electrode assembly for connection to the myocardium of the left ventricle of a patient's heart and configured for application of left ventricular pacing, LVp, and for left ventricular sensing, LVs, and
a control unit, configured to control the LV electrode assembly with respect to an application of LVp at a scheduled LV stimulation time, the control including inhibiting LVp during a left ventricle upper rate interval, LVURI, for T-wave protection, wherein a start of the LVURI is triggered by an LVs or LVp event,
wherein the control unit is further configured, if the scheduled LV stimulation time falls into the LVURI, to apply LVp when the LVURI lapses.

2. The device of claim 1, wherein a right ventricular, RV, electrode assembly for connection to the myocardium of the right ventricle of the patient's heart and configured for application of right ventricular pacing, RVp.

3. The device of claim 2, wherein the control unit is configured to control the RV electrode assembly with respect to an application of RVp at a scheduled RV stimulation time which is delayed by a ventricular-ventricular, VV, time interval with respect to the scheduled LV stimulation time so that LVp comes first, wherein the VV time interval is, in particular, 5 to 100 ms or 10 to 40 ms.

4. The device of claim 3, wherein the LVURI is less than or equal to an RV upper tracking interval, RVUTI, plus the VV time interval.

5. The device of claim 3, wherein the control unit is further configured, if the scheduled LV stimulation time falls into the LVURI, to apply LVp when the LVURI lapses only if a lapse time of the LVURI is within a predetermined time interval that includes the scheduled RV stimulation time.

6. The device of claim 1, wherein an atrial electrode assembly for connection to the myocardium of the right atrium of the patient's heart and configured for atrial sensing, As, and/or atrial pacing wherein the scheduled LV stimulation time is calculated by the control unit based on a timing of the respective atrial event.

7. The device of claim 1, wherein the LVURI is a constant that is programmable between 375 to 667 ms.

8. A method for operating an implantable medical device, comprising the steps of
sensing, by a left ventricular, LV, electrode assembly of the medical device for connection to the myocardium of the left ventricle of a patient's heart, an event, LVs;
triggering, by a control unit of the medical device, a start of a left ventricle upper rate interval, LVURI, for T-wave protection, during which an application of left ventricular pacing, LVp, through the LV electrode assembly is inhibited; and
if a scheduled LV stimulation time falls into the LVURI, applying, by the control unit, LVp when the LVURI lapses.
